(19) United States
(12) Patent Application Publication
(10) Pub. No.: US 2010/0322978 A1
(43) Pub. Date: Dec. 23, 2010

(54) MEDICAL COMPOSITION

(75) Inventors: Shouji Ooya, Kanagawa (JP); Tetsuo Hiratou, Kanagawa (JP)

Correspondence Address: SUGHRUE MION, PLLC, 2100 PENNSYLVANIA AVENUE, N.W., SUITE 800, WASHINGTON, DC, 20037, US

(73) Assignee: FUJIFILM Corporation, Tokyo (JP)

(21) Appl. No.: 12/521,485

(22) PCT Filed: Dec. 27, 2007
(86) PCT No.: PCT/JP2007/001478

§ 371 Date: Jun. 26, 2009

(30) Foreign Application Priority Data
Dec. 27, 2006 (JP) 2006-351490

(51) Int. Cl. A61K 9/00; A61K 31/337; A61K 31/7088; A61K 39/395; A61P 35/00; A61P 7/02; A61P 37/08; A61P 39/06; A61P 37/06; A61P 29/00

(52) U.S. Cl. 424/400; 514/449; 514/44 R; 424/130.1

(57) ABSTRACT

It is an object of the present invention to produce a medical composition using a multilayered biopolymer structure. The present invention provides a medical composition of multilayered biopolymer structure of at least two layers, wherein the multilayered biopolymer structure comprises different types of biopolymer structures, and at least one layer of the multilayered biopolymer structure comprises a chemical substance.

### TECHNICAL FIELD

The present invention relates to a medical composition of biopolymer structure wherein a chemical substance is encapsulated.

### BACKGROUND ART

General drug administration routes include oral administration, injection into the blood, transdermal administration, implantation into tissues, and the like. At present, oral administration and injection into the vein (injection into the blood) are mainly used.

In recent years, studies regarding such drug administration routes have vigorously been progressing. Transdermal or transpulmonary preparations, which use skin or mucosa as an administration route, are studied. These administration methods are advantageous in that they have high bioavailability and high patient compliance, in that administration is easily suspended in the case of excessive administration, and in that they facilitate administration of drugs to disable patients. Utilizing these advantages, products comprising an analgesic agent (Duragesic) and insulin products (Exubeta) have been placed on the market. These products demonstrate the effectiveness of the aforementioned administration routes. On the other hand, with regard to implantation into tissues, matrixes, in which cell growth factors and the like are contained in collagen or gelatin, are used in the regenerative medicine field (JP Patent Publication (Kokai) No. 8-325160 A (1996), JP Patent Publication (Kokai) No. 2004-203829 A, International Publication WO03/7982, etc.). In addition, JP Patent Publication (Kokai) No. 61-204125 A (1986) discloses an external preparation comprising an anticancer agent. However, this invention only relates to a monolayered preparation, and it does not describe application of an agent over time.

In general, for the treatment of focal sites, the action of a drug that is consistent with the healing process of an organism is important. That is to say, it is necessary for a drug to be appropriately administered while the type and concentration thereof are determined properly along the temporal axis of healing. However, generally, one or more types of drugs are merely encapsulated in a single substrate. JP Patent Publication (Kokai) No. 59-131355 A (1984) discloses a multiple soft capsule prepared by covering oil with gelatin and encapsulating foods in the oil layer. However, this method merely produces multilayered structures by continuation of a hydrophilic gelatin and a hydrophobic oil layer.

### Patent Document 1: JP Patent Publication (Kokai) No. 8-325160 A (1996)

### Patent Document 2: JP Patent Publication (Kokai) No. 2004-203829 A

### Patent Document 3: International Publication WO03/7982

### Patent Document 4: JP Patent Publication (Kokai) No. 61-204125 A (1986)

### Patent Document 5: JP Patent Publication (Kokai) No. 59-131355 A (1984)

### DISCLOSURE OF THE INVENTION

### Object to be Solved by the Invention

It is an object to be solved by the present invention to produce a medical composition using a multilayered biopolymer structure, which has not been studied in the prior art

### Means for Solving the Object

As a result of intensive studies directed towards achieving the aforementioned object, the present inventors have found that a medical composition that is designed to release a certain amount of a chemical substance for a long period of time or a medical composition in which the decomposition of gel by enzyme can be controlled can be provided by producing a medical composition of a biopolymer structure composed of at least 2 different types of layers comprising a chemical substance, thereby completing the present invention.

The present invention provides a medical composition of multilayered biopolymer structure of at least two layers, wherein the multilayered biopolymer structure comprises different types of biopolymer structures, and at least one layer of the multilayered biopolymer structure comprises a chemical substance.

Preferably, the biopolymer is a protein, a polysaccharide, or a derivative thereof.

Preferably, the protein comprises at least one selected from the group consisting of collagen, gelatin, albumin, laminin, casein, fibrin, fibronectin, and vitronectin.

Preferably, the origin of the protein is a human, a bovine, a swine, fish, or a gene recombinant protein.

Preferably, the protein is crosslinked.

Preferably, the protein is crosslinked by the action of transglutaminase or glutaraldehyde.

Preferably, in terms of the different types of biopolymer structures, one or more selected from among the type, concentration, content or crosslinking degree of a biopolymer, the mixing ratio of biopolymer and synthetic polymer, the type or concentration of a chemical substance encapsulated, and the type or concentration of an additive encapsulated are different.

Preferably, the form of the composition is a film.

Preferably, the chemical substance is an anticancer agent, an antiallergic agent, an antioxidant, an antithrombotic agent, an anti-inflammatory agent, an immunosuppressive agent, a nucleic acid drug, an antibody drug, a cosmetic ingredient, or a supplement ingredient.

Preferably, the chemical substance is an anticancer agent.

Preferably, the anticancer agent is 5-fluorouracil, adriamycin, paclitaxel, docetaxel, or cisplatin.

Preferably, the medical composition according to the present invention further comprises at least one additive selected from the group consisting of a coloring agent, a softening agent, a percutaneous absorption promoting agent, a moisturizing agent, a surfactant, an antiseptic agent, an aroma chemical, and a pH adjuster.

Preferably, the medical composition according to the present invention is an external preparation, a transdermal agent, a local treatment agent, an oral treatment agent, an embedded agent, a cosmetic product, or a supplement.

Preferably, the medical composition according to the present invention is used in the treatment of skin cancer, keratosis, malignant melanoma, mycosis fungoides, breast cancer, prostatic cancer, uterine cancer, vaginal cancer, scrotal cancer, or colon cancer.

### EFFECT OF THE INVENTION

According to the present invention, a structure in which the type or concentration of a chemical substance is changed can be produced without significantly changing the properties of the structure. As a result, it becomes possible to administer a chemical substance along a healing process. For example, by changing the concentration of such chemical substance, it becomes possible to continuously administer a certain concentration of the chemical substance. In addition, it is also possible to produce a wound-healing material, which suppresses inflammation in the initial stage when it is embedded in the wound site, and which then promotes healing as it becomes decomposed in a body.

### BEST MODE FOR CARRYING OUT THE INVENTION

In recent years, in addition to the development of new drugs, new administration routes have also been developed in the pharmaceutical development field. Among others, since transdermal and transmucosal administration routes are able to avoid a first pass effect and have high bioavailability, these administration routes are anticipated to be effective.

The medical composition of the present invention is a medical composition of multilayered biopolymer structure of at least two layers, which is characterized in that the multilayered biopolymer structure comprises different types of biopolymer structures, and in that at least one layer of the multilayered biopolymer structure comprises a chemical substance.

In the medical composition of the present invention, multilayered biopolymer structure of two or more layers is formed. The biopolymer structure preferably forms discontinuous layers. The term “discontinuous layers” is used herein to mean that the composition has portions in which the below-mentioned properties of the structures are discontinuously changed. Examples of such properties of the structures include the type, form, concentration, content, crosslinking degree, type of crosslinking, and crosslinking method of a biopolymer; the mixing ratio of biopolymers and synthetic polymers; the type and concentration of a chemical substance encapsulated; and the type, concentration and water content of an additive encapsulated; but examples are not limited thereto. Preferred examples of such properties of the structures include the type, concentration, crosslinking degree and type of crosslinking of the structure; and the type and concentration of the chemical substance. Particularly preferred examples include the crosslinking degree of the structure and the concentration of the chemical substance. In addition, the medical composition of the present invention may have an interface between the aforementioned biopolymer layers having different properties.

The type of the biopolymer used in the present invention is not particularly limited. A protein or a polysaccharide is preferable. The biopolymer used in the present invention is more preferably collagen, gelatin, albumin, laminin, casein, fibrin, fibronectin, or vitronectin. It is further preferably collagen, gelatin, or albumin. It is most preferably collagen or gelatin. The origin of the biopolymer is not particularly limited. The origin may be any one of a human, a bovine, a swine, a bird, or a fish, or may be a gene recombinant. Preferably, the origin is a human, a bovine, a swine, fish, or is a gene recombinant. More preferably, the origin is a bovine, a swine, fish, or is a gene recombinant. The biopolymer used as a substrate may be used singly, or several types of biopolymers may be used. Otherwise, the biopolymer may also be used in combination with a synthetic polymer.

The biopolymer used as a substrate in the present invention may be chemically modified. Chemical modification means that a portion of a functional group is allowed to bind to a low-molecular-weight or high-molecular-weight compound. The biopolymer is more preferably modified via an ester bond or an amide bond. Moreover, the biopolymer may also be crosslinked. Such crosslinking may be achieved by heat, light, a crosslinking agent, enzyme, polyion-complex, or hydrophobic interaction. Preferably, such crosslinking is achieved by a crosslinking agent or enzyme. More preferably, it is achieved by enzyme. Particularly preferable enzymes are transglutaminase and laccase. The most preferable enzyme is transglutaminase.

The transglutaminase may be derived from mammals or microorganisms. Specific examples of transglutaminase include: Activa products manufactured by Ajinomono Co., Inc.; and transglutaminases derived from mammals that are commercially available as reagents, such as guinea pig liver-derived transglutaminase, goat-derived transglutaminase and rabbit-derived transglutaminase, which are manufactured by Oriental Yeast Co., Ltd., Upstate USA Inc., Biodesign International, etc. Moreover, humanized recombinant transglutaminase may also be used.

The chemical substance used in the present invention is a physiologically active ingredient. Specific examples thereof include an external preparation, a transdermal agent, a local treatment agent, an oral treatment agent, an embedded agent, a cosmetic ingredient, and a supplement ingredient. Specific examples of the chemical substance are not particularly limited. Preferably, the chemical substance is an anticancer agent, an antiallergic agent, an antioxidant, an antithrombotic agent, an anti-inflammatory agent, an immunosuppressive agent, a nucleic acid drug, or an antibody drug. Particularly preferably, it is an anticancer agent.

Specific examples of the anticancer agent that can be used in the present invention include: pyrimidine fluoride antimetabolites (5-fluorouracil (5FU), tegafur, doxifluridine, capecitabine, etc.); antibiotics (mitomycin (MMC), adriacin (MR), etc.); purine antimetabolites (folic acid antimetabolites such as methotrexate, mercaptopurine, etc.); vitamin A active metabolites (antimetabolites such as hydroxycarbamide, tretinoin, tamibarotene, etc.); molecular-targeted agents (herceptin, imatinib mesylate, etc.); platinum-containing drugs (briplatin, randa (CDDP), paraplatin (CBDC), elplat (Oxa), aqupla, etc.); plant alkaloid drugs (topotecin, campto (CPT), taxol (paclitaxel) (PTX), taxoter (DTX), etoposide, etc.); alkylating agents (busulphan, cyclophosphamide, ifomide, etc.); antiandrogen agents (bicalutamide, flutamide, etc.); female hormone agents (fosfestrol, chlormadinone acetate, estramustine phosphate, etc.); LH-RH agents (leuplin, zoladex, etc.); antiestrogen agents (tamoxifen citrate, tremifene citrate, etc.); aromatase inhibitors (fadrozole hydrochloride, anastrozole, exemestane, etc.); progestational hormone agents (medroxyprogesterone acetate, etc.); and BCG. However, examples are not limited thereto in the present invention.

Specific examples of the antiallergic agent used in the present invention include, but are not limited to, mediator release-suppressing agents such as disodium cromoglycate and tranilast, histamine H1 antagonists such as ketotifen fumarate and azelastine hydrochloride, thromboxane inhibitors such as ozagrel hydrochloride, leukotrien antagonists such as pranlukast, and suplatast tosilate.

Specific examples of the antioxidant used in the present invention include, but are not limited to, vitamin C and the derivative thereof, vitamin E, kinetin, α-lipoic acid, coenzyme Q10, polyphenol, SOD, phytic acid, and astaxanthin.

Specific examples of the antithrombotic agent used in the present invention include, but are not limited to, aspirin, ticlopidine hydrochloride, cilostazol, and warfarin potassium.

Specific examples of the anti-inflammatory agent used in the present invention include, but are not limited to, compounds selected from among azulene, allantoin, lysozyme chloride, guaiazulene, diphenhydramine hydrochloride, hydrocortisone acetate, predonisolone, glycyrrhizic acid, glycyrrhetic acid, glutathione, saponin, methyl salicylate, mefenamic acid, phenylbutazone, indomethacin, ibuprofen and ketoprofen, the derivatives thereof, and the salts thereof, and any one selected from among plant extracts selected from among Scutellariae radix extract, Artemisia capillaris extract, balloonflower (Platycodon grandiflorus) extract, Armeniacae semen extract, gardenia extract, Sasa veitchii extract, gentiana extract, comfrey extract, white birch extract, mallow extract, Persicae semen extract, peach leaf extract, and Eriobotryae folium extract.

Specific examples of the immunosuppressive agent used in the present invention include, but are not limited to, rapamycin, tacrolimus, cyclosporine, predonisolone, methylpredonisolone, micophenolate mofetil, azathioprine, and mizoribine.

Specific examples of the nucleic acid drug used in the present invention include, but are not limited to, antisense, ribozyme, siRNA, an aptamer, and a decoy nucleic acid.

In addition to these chemical substances, various types of additives may also be added. Examples of the additive that can be used in the present invention include: moisturizing agents (for example, agar, diglycerin, distearyldimonium hectorite, butylene glycol, polyethylene glycol, propylene glycol, sodium hyaluronate, hexylene glycol, Coix lachrma-jobi extract, and Vaseline); softening agents (for example, glycerin and mineral oil); emollient ingredients (surfactants, etc.) (for example, isopropyl isostearate, polyglyceryl isostearate, isotridecyl isononanoate, octyl isononanoate, oleic acid, glyceryl oleate, cacao butter, cholesterol, mixed fatty acid triglyceride, dioctyl succinate, sucrose acetate stearate, cyclopentasiloxane, sucrose distearate, octyl palmitate, octyl hydroxystearate, arachidyl behenate, sucrose polybehenate, polymethylsilsesquioxane, myristyl alcohol, cetyl myristate, myristyl myristate, hexyl laurate, and cremophor); percutaneous absorption promoting agents (for example, ethanol, isopropyl myristate, citric acid, squalane, oleic acid, menthol, N-methyl-2-pyrrolidone, diethyl adipate, diisopropyl adipate, diethyl sebacate, diisopropyl sebacate, isopropyl palmitate, isopropyl oleate, octyldodecyl oleate, isostearyl alcohol, 2-octyl dodecanol, urea, vegetable oil, and animal oil); antiseptics (for examples, benzoic acid, sodium benzoate, ethyl parahydroxybenzoate, potassium sorbate, sodium sorbate, sorbic acid, sodium dehydroacetate, and methyl parahydroxybenzoate); coloring agents (kaoline, carmine, ultramarine blue, chrome oxide, and iron oxide); aroma chemicals; and pH adjusters (for example, sodium citrate, sodium acetate, sodium hydroxide, potassium hydroxide, and phosphoric acid).

The form of the medical composition is not particularly limited. The form may be a gel, a sponge, a film, a nonwoven fabric, a fiber (a tube), and a particle, for example. The medical composition may have any shape. A pyramidal, conical, prismatic, columnar, spherical or fusiform structure, and a substrate produced using any given mold may be used. Moreover, the composition may be coated with various types of sheets such as a waterproof sheet. Examples of the material of such sheet include polyethylene, polypropylene, and polyvinylidene chloride.

The intended use of the external preparation is not particularly limited. The external preparation is used, for example, for skin cancer, keratosis, malignant melanoma, mycosis fungoides, breast cancer, prostatic cancer, uterine cancer, vaginal cancer, scrotal cancer, and colon cancer. Preferably, it is used for skin cancer and keratosis. Moreover, a preferred intended use of the composition of the present invention used as an external preparation is a transdermal agent. Furthermore, the external preparation may also be embedded in tissues, as necessary. For example, in order to maintain the function of a site that has been eliminated by operation, the external preparation of the present invention may be embedded in such eliminated tissue site, or it may cover a significantly damaged skin area. When an external preparation has high affinity for living bodies, it may act to help the repair of peripheral tissues.

Since the present composition comprises a biopolymer, it is also useful as an oral agent. When a chemical substance that is easily decomposed in stomach is used, for example, it is feared that the chemical substance would be decomposed before reaching the intestine. In the case of the agent of the present invention, it is also possible to design an agent that is decomposed in the intestine. Thus, it becomes possible to produce an oral agent for a chemical substance that is generally hardly administered via oral administration. For example, it becomes possible to produce an oral agent for an antibody drug or a protein drug such as insulin.

The present invention will be more specifically described in the following examples. However, the present invention is not limited by the examples.

### EXAMPLES

### Example 1

### Production of 5-FU-Encapsulated Multilayered Gelatin Gel

PBS solution (10 mL) containing acid-treated gelatin (10%), 5-FU (1, 2, 3, 4, 5, 6, 7, 8, 9, or 10 mg/mL) and transglutaminase (0.8%; Activa TG-S; manufactured by Ajinomoto Co., Inc.) was separately coated onto a polystyrene plate (10 cm×10 cm; thickness: 1 mm). It was left at rest at 25° C. for 17 hours to produce 10 types of 5-FU-encapsulated crosslinked gelatin gels. The films were successively laminated one on another to produce 5-FU-containing gelatin gel of 10 layers (thickness: 1 cm).

### Comparative Example 1

### 5-FU-Encapsulated Monolayered Gelatin Gel

PBS solution (100 mL) containing acid-treated gelatin (PSP gelatin; manufactured by Nippi, Inc.; 10%), 5-FU (5.5 mg/mL) and transglutaminase (0.8%; Activa TG-S; manufactured by Ajinomoto Co., Inc.) was poured into a mold on a polystyrene plate (10 cm×10 cm; thickness: 1 cm). It was left at rest at 25° C. for 17 hours to produce a monolayered 5-FU-containing gelatin gel (thickness: 1 cm).

### Example 2

PBS solution (10 mL) containing acid-treated gelatin (10%), 5-FU (5 mg/ml) and transglutaminase (Activa TG-S; manufactured by Ajinomoto Co., Inc.) (0.1, 0.2, 0.3, 0.4, 0.5, 0.6, 0.7, 0.8, 0.9, or 1.0%) was separately coated onto a polystyrene plate (10 cm×10 cm; thickness: 1 mm). It was left at rest at 25° C. for 17 hours to produce 10 types of 5-FU-encapsulated crosslinked gelatin gels having different crosslinking degrees. These films were successively laminated one on another to produce 5-FU-containing gelatin gel of 10 layers (thickness: 1 cm).

### Comparative Example 2

PBS solution (100 mL) containing acid-treated gelatin (10%), 5-FU (5 mg/mL) and transglutaminase (Activa TG-S; manufactured by Ajinomoto Co., Inc.) (0.55%) was poured into a mold on a polystyrene plate (10 cm×10 cm; thickness: 1 cm). It was left at rest at 25° C. for 17 hours to produce a monolayered 5-FU-containing gelatin gel.

### Example 3

### Behavior of 5-FU Releasing from 5-FU-Encapsulated Multilayered Gelatin Gel

A portion of 1 cm×1 cm×1 cm was cut out of each of the gels produced in Example 1 and Comparative example 1, and it was then fit into the bottom of a mold (1 cm×1 cm; thickness: 50 cm) (the multilayered gel was placed such that the 5-FU concentration increased from the PBS-contact surface to the bottom). Thereafter, it was added in PBS (50 mL), and the amount of 5-FU released after a certain period of time was measured by HPLC. As a result, it was found that a large amount of 5-FU was released from the monolayered gel (Comparative example 1) for the first several hours, that the release amount decreased over time, and that 90% of 5-FU was released for 1 day. On the other hand, in the case of the multilayered gel (Example 1), the amount of 5-FU released was smaller than that of the monolayered gel for the first several hours. However, approximately 90% of 5-FU was released for 1 day in the case of the multilayered gel. The release of 5-FU from the multilayered gel was slow in the initial stage, and thus a decrease in the releasing rate over time was significantly suppressed. That is to say, by creating a multilayered gel, it becomes possible to design to release a certain amount of agent for a long period of time.

### Example 4

### In Vitro Degradability of 5-FU-Containing Gelatin Gel

A portion of 1 cm×1 cm×1 cm was cut out of each of the gels produced in Example 2 and Comparative example 2, and it was then fit into the bottom of a mold (1 cm×1 cm×10 cm) (the multilayered gel was placed such that the crosslinking degree increased from the solution-contact surface to the bottom). Thereafter, 5 mL of thermolysin (5 μM), an enzyme for decomposing the gelatin, was added thereto, and it was then left at rest at room temperature. Both types of gels were decomposed over time. In the case of the multilayered gel (Example 2), the initial decomposition rate was fast, and half of the gel was decomposed for approximately 3 hours. It took 8 hours to decompose the multilayered gel as a whole. On the other hand, in the case of the monolayered gel (Comparative example 2), the gel exhibited a linear decomposition behavior. It took 4.5 hours to decompose half of the gel, and it took approximately 9 hours to terminate decomposition of the gel. Thus, by creating a multilayered gel in which individual layers had different crosslinking degrees, it became possible to control the decomposition of the gel by the action of the enzyme.

### Example 5

### Production of Paclitaxel-Containing Gelatin-Polylactic Acid-Albumin Mixed Film

1,1,1,3,3,3-hexafluoro-2-propanol (HFIP) solutions (10 mL each) containing 1 g of any one of acid-treated gelatin, polylactic acid or albumin, were prepared. Thereafter, 1 mg of paclitaxel was dissolved in each solution. First, a gelatin solution containing paclitaxel was poured into a mold (10 cm×10 cm) made of silicon, and it was then humidified for 1 day (50° C.; humidity: 100%) to produce a paclitaxel-encapsulated gelatin film. Thereafter, a paclitaxel-containing polylactic acid solution was poured onto the film, and it was then humidified for 1 day (50° C.; humidity: 100%) to produce a paclitaxel-encapsulated polylactic acid film on the gelatin film (a gelatin-polylactic acid mixed film). Thereafter, a paclitaxel-containing albumin solution was further poured into the gelatin-polylactic acid mixed film, and it was then humidified for 1 day (50° C.; humidity: 100%) to produce a paclitaxel-containing albumin film on the gelatin-polylactic acid mixed film (a gelatin-polylactic acid-albumin mixed film). By the aforementioned operations, a paclitaxel-containing gelatin-polylactic acid-albumin mixed film could be produced.

The thus produced film is a highly-functional film, in which 3 layers having significantly different properties, namely, a cell-adhesion gelatin layer, a high-strength polylactic acid layer, and a cell-non adhesion albumin layer, are adhered to one another. That is to say, by the aforementioned method, a chemical substance-encapsulated protein structure, in which substrates had different chemical, physical and structural properties, could be produced.

### INDUSTRIAL APPLICABILITY

According to the present invention, a structure in which the type or concentration of a chemical substance is changed can be produced without significantly changing the properties of the structure. In addition, there can also be produced a structure whose chemical, physical, and structural properties are adjusted for desired uses.

1. A medical composition of multilayered biopolymer structure of at least two layers, wherein the multilayered biopolymer structure comprises different types of biopolymer structures, and at least one layer of the multilayered biopolymer structure comprises a chemical substance.

2. The medical composition according to claim 1, wherein the biopolymer is a protein, a polysaccharide, or a derivative thereof.

3. The medical composition according to claim 2, wherein the protein is at least one selected from the group consisting of collagen, gelatin, albumin, laminin, casein, fibrin, fibronectin, and vitronectin.

4. The medical composition according to claim 2, wherein the origin of the protein is a human, a bovine, a swine, fish, or a gene recombinant protein.

5. The medical composition according to claim 2, wherein the protein is crosslinked.

6. The medical composition according to claim 5, wherein the protein is crosslinked by the action of transglutaminase or glutaraldehyde.

7. The medical composition according to claim 1, wherein, in terms of the different types of biopolymer structures, one or more selected from among the type, concentration, content or crosslinking degree of a biopolymer, the mixing ratio of biopolymer and synthetic polymer, the type or concentration of a chemical substance encapsulated, and the type or concentration of an additive encapsulated are different.

8. The medical composition according to claim 1, wherein the form of the composition is a film.

9. The medical composition according to claim 1, wherein the chemical substance is an anticancer agent, an antiallergic agent, an antioxidant, an antithrombotic agent, an anti-inflammatory agent, an immunosuppressive agent, a nucleic acid drug, an antibody drug, a cosmetic ingredient, or a supplement ingredient.

10. The medical composition according to claim 9, wherein the chemical substance is an anticancer agent.

11. The medical composition according to claim 10, wherein the anticancer agent is 5-fluorouracil, adriamycin, paclitaxel, docetaxel, or cisplatin.

12. The medical composition according to claim 1, which further comprises at least one additive selected from the group consisting of a coloring agent, a softening agent, a percutaneous absorption promoting agent, a moisturizing agent, a surfactant, an antiseptic agent, an aroma chemical, and a pH adjuster.

13. The medical composition according to claim 1, which is an external preparation, a transdermal agent, a local treatment agent, an oral treatment agent, an embedded agent, a cosmetic product, or a supplement.

14. The medical composition according to claim 1, which is used in the treatment of skin cancer, keratosis, malignant melanoma, mycosis fungoides, breast cancer, prostatic cancer, uterine cancer, vaginal cancer, scrotal cancer, or colon cancer.

15. The medical composition according to claim 3, wherein the origin of the protein is a human, a bovine, a swine, fish, or a gene recombinant protein.
